(19) United States
(12) Patent Application Publication
(10) Pub. No.: US 2006/0172412 A1
(43) Pub. Date: Aug. 3, 2006

(54) Support device for cells culture

(75) Inventors: Eric Perrier, Les Cotes D'Arey (FR); Odile Damour, Saint Genis Laval (FR); Valerie Andre, Ampuis (FR)

Correspondence Address: MERCHANT & GOULD PC, P.O. BOX 2903, MINNEAPOLIS, MN, 55402-0903, US

(73) Assignees: COLETICA, Lyon (FR); Centre National de la Recherche Scientifique, Paris (FR)

(21) Appl. No.: 11/127,898

(22) Filed: May 12, 2005

(30) Foreign Application Priority Data
Feb. 2, 2005 (FR) 05 01051

(51) Int. Cl. C12M 1/22; C12M 1/12

(52) U.S. Cl. 435/297.5; 435/305.1; 435/305.4

(57) ABSTRACT

a cell culture device is disclosed having at least one cell-supporting layer of porous material obtained by dehydration of an aqueous gel which is poured directly either into said well bottom or into the bottom of a cradle or insert of a size which is adapted for being insertable into the volume of the well, thereby enabling by a robot or an automaton itself commanded by you computer.

### FIELD OF THE INVENTION

The invention relates to a culture device which enables a better reproducibility des cultures.

### DESCRIPTION OF PRIOR ART

The closest state of the art is constituted by the document EP 0 296 078.

This document describes novel biomaterials which are based on a mixture of collagen, chitosan and glycosaminoglycan. This biomaterial is notably used as a support for cell proliferation, notably in the case of grafting onto burned patients. In the case of a manufacture of a support for cell proliferation, this support is in general formed by a support film which is obtained by drying of a gel poured into flasks, Petri dishes or multi-well plates (vide Example 5, page 14).

An embodiment is also provided which is described in Example 1, and which is notably for making a dermis, an essential component of an artificial skin, according to which the gel formed by a homogeneous solution is freeze-dried in industrial freeze-driers, or is dehydrated by heat dehydration in ovens which are optionally under vacuum.

Example 5 also provides a variant according to which a freeze-dried or dehydrated matrix can be used in vitro for making three-dimensional cultures of cells (fibroblasts, keratinocytes, chondrocytes, etc.) (page 14, lines 8 to 11). In this case, as described in Example 1, the freeze-drying takes place in an industrial freeze-dryer or the heat dehydration takes place in an oven which is optionally under vacuum, and the substrate obtained, which is constituted of a sponge, must first of all be cut out to the size necessary and then be transferred into a flask, a Petri dish or a multi-well plate for example, in order to carry out said culture in vitro.

### AIMS OF THE INVENTION AND TECHNICAL PROBLEMS TO BE SOLVED

A main aim of the invention is to solve the novel technical problem which relates to the preparation of substrates of cultures, having a better reproducibility.

A main aim of the invention is also to solve the novel technical problem which consists in reducing the number of steps of the method of preparation of the culture plates comprising a porous medium which is intended for receiving the cells.

Another main aim of the present invention is to solve the novel technical problem which consists in providing a method of preparing a sterile culture substrate(s) support device in a reproducible, safe and reliable manner on a large scale for an industrial and medical use, notably so as to enable the making of a high-output screening.

Another main aim of the present invention is to solve the novel technical problem which consists in providing a solution of a culture substrate(s) support device which can be used on a roboticizable or automatable platform, preferably enabling the substrates to be positioned so that the device for injecting or for aspirating the culture medium can be placed automatically, notably by being commanded by a robot or an automaton, e.g. itself commanded by a computer, and this in a reproducible manner.

Finally, a main aim of the present invention is to solve the novel technical problem which consists in providing a solution of a culture substrate support device which can be used in various types of support, notably making use of detachable inserts which are compatible with an automatization or roboticization for the positioning of the insert, with a view to an automated culture and/or an automated screening.

### SUMMARY OF THE INVENTION

According to a first aspect, the invention relates to a cell culture device, comprising at least one cell culture well intended for receiving cells to be cultivated with their culture medium, comprising a bottom, characterised in that it contains at least one cell-supporting layer of porous material, which is obtained by dehydration, preferably by freeze-drying or by heat dehydration, of an aqueous gel which is poured directly either into said well bottom, or into the bottom of a cradle or insert of a size which is adapted for being insertable into the volume of the well.

According to an advantageous feature of the invention, the device is characterised in that at least the bottom of the well or of the cradle or insert containing the layer of porous material is sterilised after said dehydration, and preferably in a watertight packaging.

According to another advantageous feature of the invention, the device is characterised in that the complete culture device is sterilised, preferably in a watertight packaging.

According to yet another advantageous feature of the invention, the sterilisation mentioned above is selected from the group consisting of a sterilisation by irradiation, preferably with beta or gamma rays, or of a treatment with a sterilising gas such as ethylene oxide.

According to another advantageous feature of the invention, the device is characterised in that it preferably comprises an element maintaining in position at least the peripheral edge of the layer of porous material by producing an anti-retraction effect of the layer of porous material and preferably also ensuring the watertightness at the peripheral interface of said layer of porous material and of the internal side wall facing it at the bottom.

Within the context of the invention, this watertightness at the periphery is advantageously provided for in order to avoid exchanges of material via the periphery, i.e. to avoid a communication via the side edge of the layer of porous material between substances which are deposited on the upper surface of the layer of porous material and the culture medium or the cells which can be present in the layer of porous material.

According to a particular embodiment of the invention, the position-maintaining element mentioned above comprises an annular ring of a size which is sufficient to take support on the peripheral edge of the layer of porous material.

According to another advantageous feature of the invention, the device is characterised in that said bottom of the cradle is detachable and joinable to the cradle, which is itself joinable to the well.

According to yet another advantageous feature of the invention, the device is characterised in that said detachable bottom of the cradle is joined by fitting with slight forcing or snap-engagement to the well, in thus ensuring the watertightness safely and reliably.

According to yet another advantageous feature of the invention, the device is characterised in that said detachable bottom of the cradle is joined to the outside of the well or of the cradle, the lower edge of the side wall of the well or of the cradle thus constituting an element maintaining in position the peripheral edge of said layer of porous material.

According to yet another advantageous feature of the invention, the device is characterised in that at least one part of the internal wall of the bottom or of the cradle is treated physically or chemically or biologically, or a combination of these, for promoting the cell culture, e.g. with a coating which promotes the adhesion and/or the proliferation of the cells.

Within the context of the invention, any physical or chemical method can be used which modifies the overall ionic charge of the material of the wall, advantageously a plastic material and/or a coating of the wall can be used with any biological molecule which promotes the adhesion and/or the proliferation of the cells, such as collagen, fibronectin, laminin, etc.

According to yet another advantageous embodiment of the invention, the device is characterised in that the bottom of the well or of the cradle is made from an inert support material selected from the group consisting of a synthetic material, material based on nitrocellulose, a material based on polyamide such as a “nylon”, a material based on polytetrafluoroethylene or teflon, a material based on polycarbonate, a semi-permeable material based on polyethylene or polyethylene terephthalate (PET), a material based on a polyester, e.g. a cellulose polyester, notably an acetate, as material based on a semi-permeable Biopore-CM membrane, or even polyvinylpyrrolidone.

The expression “material based on” is understood as meaning a material which comprises or is constituted essentially of, or solely of, the material considered.

According to another advantageous embodiment of the invention, the device is characterised in that it comprises a plurality of cradles or inserts per culture well.

According to another advantageous variant of this embodiment, this device also comprises a lid which is provided with as many orifices as cradles or inserts, each orifice enabling receiving and maintaining in position a cradle or insert.

According to a particular embodiment of the invention, the gel mentioned above, which is poured directly into the inside of the well or of the cradle, comprises collagen.

According to an advantageous feature, the gel comprises a mixture of collagen and at least one polysaccharide.

According to yet another advantageous embodiment, the gel comprises a mixture of collagen, at least one polysaccharide and chitosan, which is optionally modified, e.g. in having a degree of acylation, preferably acetylation, which is regulated as a function of the application sought after, various degrees of acetylation being well known to the person skilled in the art and are in particular described in the European document EP 0296 078 mentioned above.

In a second aspect, the invention relates also to the use of the device, as defined above or in the following description, on a roboticizable or automatable platform, preferably enabling the substrates to be positioned so that a device for injecting or for aspirating the culture medium can be placed automatically, notably by being commanded by a robot or an automaton e.g. itself commanded by a computer.

It is understood that by virtue of the invention, the various technical problems set forth above are indeed solved, in a simple, safe and reliable manner, which is reproducible on an industrial and medical scale, particularly on an industrial and pharmaceutical scale.

Other aims, features and advantages of the invention will appear clearly in the light of the explanatory description which follows made with reference to several currently preferred embodiments of cell culture devices according to the invention, which are given simply as an illustration and which in no way limit the scope of the invention. It is to be noted that the annexed Figures make up an integral part of the invention and that any feature which appears to be novel from the description taken in its entirety and from the annexed Figures making up the present description, with respect to any state of the art, is claimed as such in its function and therefore as a general means.

### DESCRIPTION OF THE FIGURES

FIG. 1 represents an exploded perspective view of a first embodiment of a cell culture support device according to the present invention which here comprises an individual culture well, which is provided with an element maintaining in position a layer of porous material;

FIG. 2 represents the embodiment of FIG. 1 in an assembled position, with the exception of the closing element;

FIG. 3 represents a cross-section view along the section line III-III of FIG. 2;

FIG. 4 represents an embodiment variant of the first embodiment which is represented in FIGS. 1 to 3, of a cell culture support device according to the present invention which here comprises a multi-well culture plate

FIG. 5 represents an exploded perspective view of a second embodiment of a cell culture support device according to the present invention which here comprises a cradle which can be inserted into an individual culture well;

FIG. 6 represents this second embodiment of FIG. 5 in an assembled position, with the exception of the closing element;

FIG. 7 represents a cross-section view along the section line VII-VII of FIG. 6;

FIG. 8 represents a variant of the second embodiment of a cell culture support device according to the present invention, within the context of a multi-well culture plate;

FIG. 9 represents a third embodiment of the cell culture support device according to the present invention, within the context of a variant of the combination of a detachable well and a position-maintaining element which is constituted here by an annular position-maintaining ring, within the context of a multi-well culture plate.

FIG. 10 represents a fourth embodiment of a cell culture support device according to the present invention, comprising a common culture well of a volume which is sufficient for receiving a plurality of cradles or inserts; and

FIG. 11 represents a partial longitudinal section view of the culture device which shows a plurality of cradles or inserts which are maintained in a plurality of orifices provided in a lid, which enables an advantageous use within the context of a roboticized platform, according to which a pipette can be inserted in an automated way for a placing in or a removal of the culture medium.

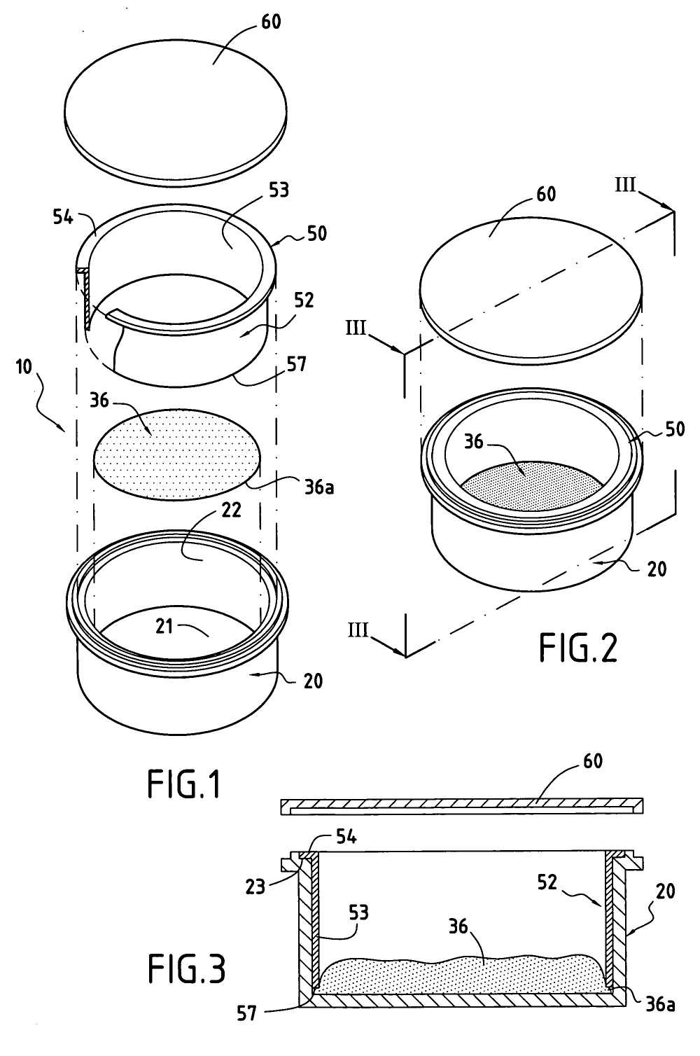

With reference to FIGS. 1 to 3, a first embodiment is represented of a cell culture device which is represented by the general reference number 10, comprising at least one cell culture well 20, e.g. of approximately cylindrical shape, intended for receiving cells to be cultivated (which are naturally not represented) with their culture medium, comprising a bottom 21, characterised according to the present invention in that it contains at least one layer 36 of cell-supporting porous material, which is obtained by dehydration, preferably by freeze-drying or by heat dehydration, of an aqueous gel which is poured directly, according to this first embodiment, into said bottom 21 of the well. The gel thus poured in and dehydrated, forms a film or a sponge at the bottom of the well.

According to an advantageous embodiment of the invention, the device is characterised in that at least the bottom 21 of the well 20 containing the layer 36 of porous material is sterilised after said dehydration, and preferably in a watertight packaging (not represented here).

According to yet another advantageous embodiment of the invention, the device is characterised in that the complete culture device 10 is sterilised, preferably in a watertight packaging (not represented).

According to a particularly advantageous embodiment variant, the sterilisation is selected from the group consisting of a sterilisation by irradiation, preferably with beta or gamma rays, or a sterilisation by treatment with a sterilising gas such as ethylene oxide.

According to yet another advantageous embodiment of the invention, this device is characterised in that it comprises an element 50 maintaining the position of at least the peripheral edge 36a of layer 36 of porous material, by producing an anti-retraction effect of layer 36 of porous material and preferably also ensuring the watertightness at the peripheral interface of said layer of porous material and of the internal side wall 22 facing it of well 20.

As the person skilled in the art will understand easily, within the context of the invention, this watertightness at the periphery is advantageously provided for in order to avoid exchanges of material via the periphery, i.e. to avoid a communication via the side edge of the layer of porous material between substances which are deposited on the upper surface of the layer of porous material and the culture medium or the cells which can be present in the layer of porous material.

According to a particular embodiment of the invention, the position-maintaining element 50 mentioned above comprises an annular ring 52 of a size which is sufficient to take support on the peripheral edge 36a of layer 36 of porous material and to flatten at least partially this peripheral edge 36a so as to ensure an anti-retracting effect of the layer 36 of porous material, as is indeed shown in the cross-section of FIG. 3.

In the embodiment represented, the annular ring 52 has a cylindrical internal wall 53 which will in practice also constitute an effective internal wall of the culture well 20, overhanging the porous layer 36. The annular ring 52 can advantageously have, on its upper part, a stopping element 54, such as a flange, also for facilitating the removal of maintaining element 50. The stopping element 54 can be provided to come to position on a shoulder 23 of the wall 22 of the well 20. A classical closing element 60, such as a lid, can also be provided.

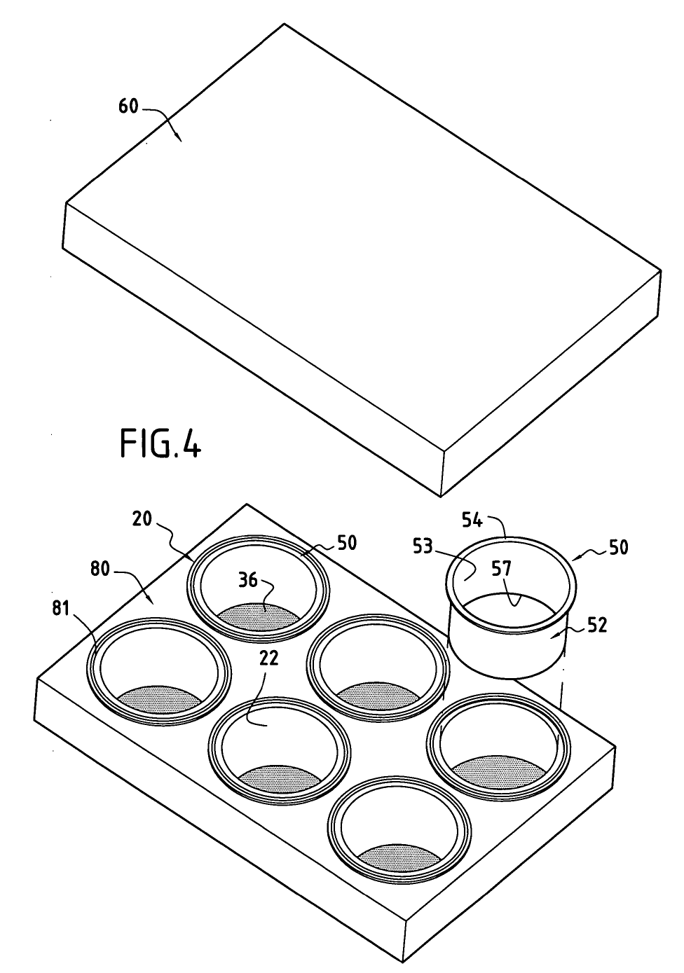

With reference to FIG. 4, an embodiment variant of the first embodiment of FIGS. 1 to 3 is shown, according to which a plate 80 having multi-openings 81 is provided for receiving a plurality of culture devices 10 with their wells 20 each having a layer 36 of porous material and for which a plurality of maintaining elements 50 is provided, as is well-understandable for the person skilled in the art.

According to another embodiment variant of the invention, it is clear that the plate 80 can integrate the plurality of the culture wells 20 in which the layers 36 of porous material can be formed initially in situ by dehydration, preferably by freeze-drying or by heat dehydration, of a gel which is poured directly into the bottom 21 of each well 20.

This plate 80 can advantageously be covered by a lid-forming element 60, enabling the culture in avoiding contaminations, as is also well-known to the person skilled in the art.

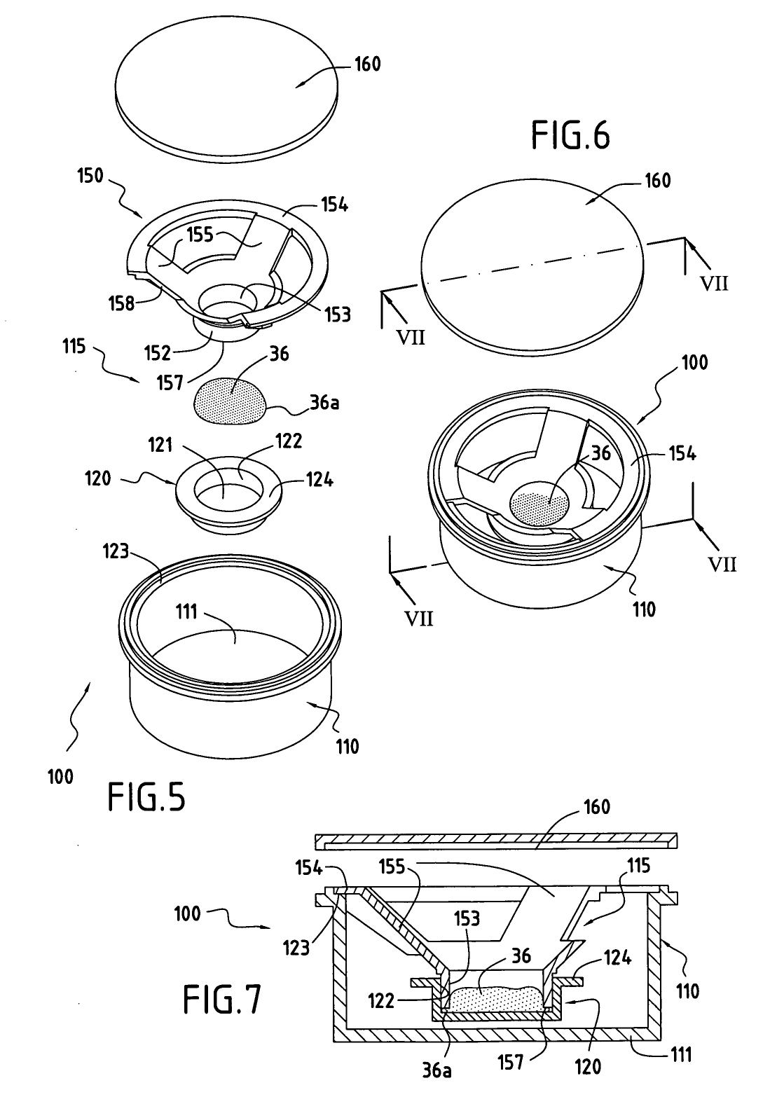

According to a second embodiment which is represented in FIGS. 5 to 7, similarly to that of FIGS. 1 to 3, and for which the reference numbers of the similar or identical pieces keep the same reference number or it is increased by 100.

It is observed that here, the complete culture device, which is here referenced 100, firstly comprises a first lower piece 110 which defines a first well, which is called external culture well per se, which comprises, on its upper edge, a shoulder 123 which is to the shoulder 23 represented in FIG. 3; and, secondly, a second piece 115 which is here named a cradle, and which is composed, according to this embodiment represented, of two distinct elements, namely:

a first element, called a lower element 120, which forms a second well, called internal culture well, similar in function to the culture well 20 which is represented in FIGS. 1 to 3, which also comprises a wall which forms the bottom 121, an internal side wall 122, and an upper edge, which is here in the form of a flange, 124 which is intended more particularly here to facilitate the prehension of this first lower element 120; and

a second element, called upper element 150, which also functions as a maintenance element, and which therefore has a function which is similar to the maintenance element 50 of the first embodiment of FIGS. 1 to 3, which here comprises an approximately cylindrical lower part 152, of function which is similar to the cylindrical part of the annular ring 52 of the maintenance element 50 and the internal surface 153 of which is similar to the internal surface 53 of the annular ring 52; an intermediate part here formed by linking arms 155 which link the cylindrical part 152 to an upper part defining an upper edge which is here in the form of flange 154, which is similar to flange 54 of maintenance element 50 of the first embodiment of FIGS. 1 to 3.

This second element 150 also comprises a cut out or gap 158 in the upper part which forms the upper edge 154, advantageously extending here over all the distance which separates the two adjacent linking arms 155, so as to give a space which is sufficient for the passage of a tubular part of a pipette, or multi-channel pipette point, or of an addition or pouring in cannula, or of an automatic removal cannula, which enables the distribution and the removal of a solution of the culture medium in the well-forming element 120 in which layer 36 of porous material has initially been formed in situ.

It is in fact noted that according to this second embodiment of the invention, said first element 120 which constitutes in practice a culture well is detachable, as is well visible in FIG. 5 and is joinable at least provisionally with the second element 150.

Thus, it is possible to pour the initial gel directly into the well defined by bottom 121 and side wall 122 of these first elements 120, and to then place these first elements 120 in freeze-driers or in ovens, optionally under vacuum, so as to form layer 36 of porous material in situ.

Then, these first elements 120 can be combined with the second elements 150 which are provided such that the lower annular cylindrical part 152 comes to take support on the peripheral edge 36a of the layer 36 of porous material to compress it and to ensure an anti-retraction effect of this peripheral edge 36a of the layer of porous material 36, as is well visible in FIG. 7 in a manner which is similar to the first embodiment as represented in FIG. 3.

Thus, according to an advantageous embodiment of the invention, the first element 120 which forms the well bottom of the cradle 115 is joined by fitting with slight forcing or snap-engagement to the second element 150, which in the case of the cradle 115 is formed by the cylindrical wall 152.

According to yet another advantageous embodiment of the invention, said bottom 121 of the first element 120 is joined to the outside of the cylindrical part 152 of the second upper element 150 the internal wall 54 of which in practice also defines a wall of the well of the cradle 115, whereas the lower edge 157 of the cylindrical part 152 making up a part of the cradle constitutes an element 150 to maintain in position the peripheral edge 36a of said layer 36 of porous material.

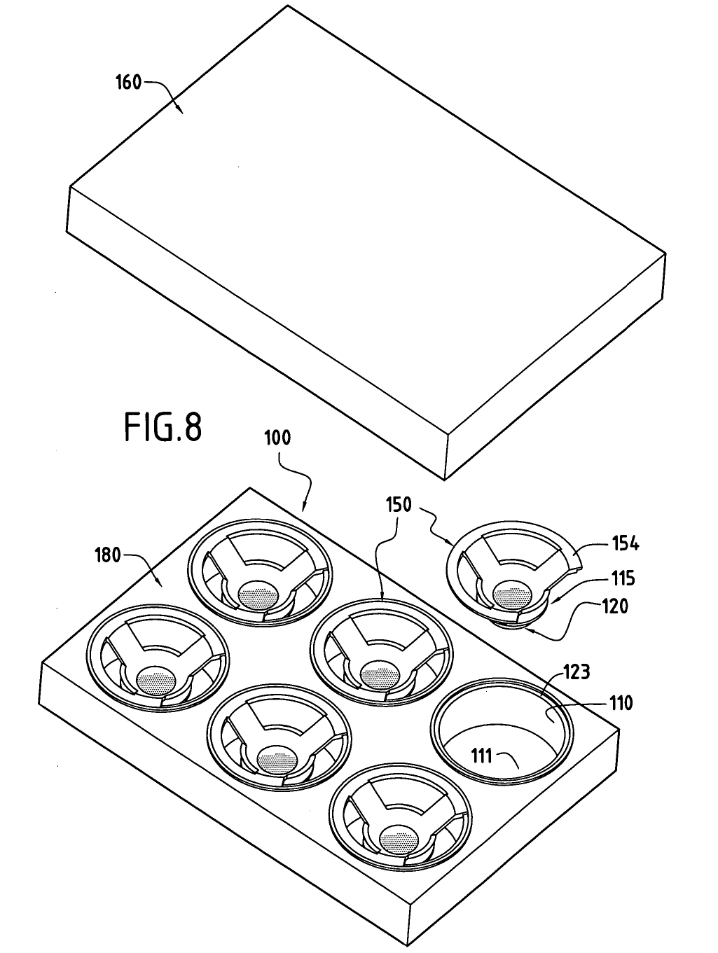

With reference to FIG. 8, representation is made in a manner similar to FIG. 4 of a plate, which here bears the reference number 180, of multi-openings to receive a plurality of culture devices 100 with their cradles 115 comprising the wells 120, each having a layer 36 of porous material and for which a plurality of maintenance elements 150 is provided, as is well visible in FIG. 8 and well understandable for the person skilled in the art.

According to another embodiment variant of the invention represented here in FIG. 8, it is clear that the plate 180 can integrate the plurality of the external culture wells 110 comprising a bottom 111 in the volume of which the cradles 150 can be arranged, which comprise the first elements 120 defining the internal wells defined by the walls forming bottom 121 and side 122, containing the layers 36 of porous material and having been formed initially in situ by dehydration, preferably by freeze-drying or by heat dehydration, of a gel which is poured directly into the bottom 21 of each well 20.

It is understood that this culture device structure is novel per se and non-obvious to the person skilled in the art.

This plate 180 can be advantageously covered with a lid forming element 160, to enable the culture in avoiding inopportune contaminations, in a manner which is similar to the embodiment of FIG. 4.

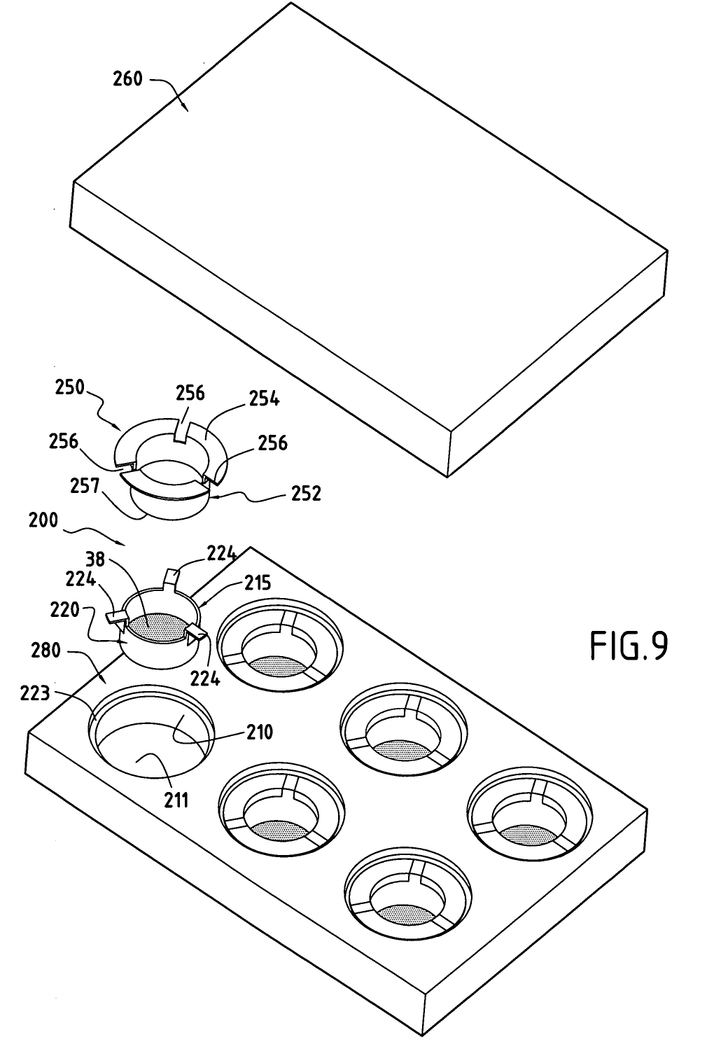

Finally, with reference to FIG. 9, another embodiment variant is represented for which the reference numbers have been further increased by 100, of a plate 280 comprising a plurality of cavities having a side wall 210 with a shoulder on its upper edge 223, and a wall forming bottom 211, defining external wells in the volume of which a plurality of culture devices 200 according to the present invention can be inserted, but according to which here the cradle 215 is made as a single-piece and constitutes as it were an insert defining the internal culture well 220 per se which can be here of a bigger size than the lower part forming well 120 of the embodiment of FIGS. 5 to 8.

According to this variant of the third embodiment, flaps 254, which are similar to flange 54 or 154 of the preceding embodiments, are provided on the cradle or insert 215, to come to take support on the upper surface forming shoulder 223 of the plate 280 comprising multiple openings 281 with a bottom 221, which are well visible in FIG. 9. As in the preceding embodiments, these cradles or inserts 215 contain a layer 38 of porous material which is formed in situ from a gel, by dehydration, preferably carried out by freeze-drying or by heat dehydration.

According to the invention, the culture device 200 also comprises a position-maintaining means 250, which is similar to means 50, FIGS. 1 to 4, and to means 150, FIGS. 5 to 8, advantageously comprising an annular ring 252 having a cylindrical part the lower edge 257 of which will have the function of anti-retraction of the peripheral edge 36a of the layer 36 of porous material, as for the preceding embodiments.

The annular ring 252 advantageously also comprises a flange 254 comprising hollows 256, which correspond to flaps 224.

The person skilled in the art will understand easily that the invention can be implemented with various embodiments, without leaving its context. Further, these embodiments make up an integral part of the invention and are claimed as such.

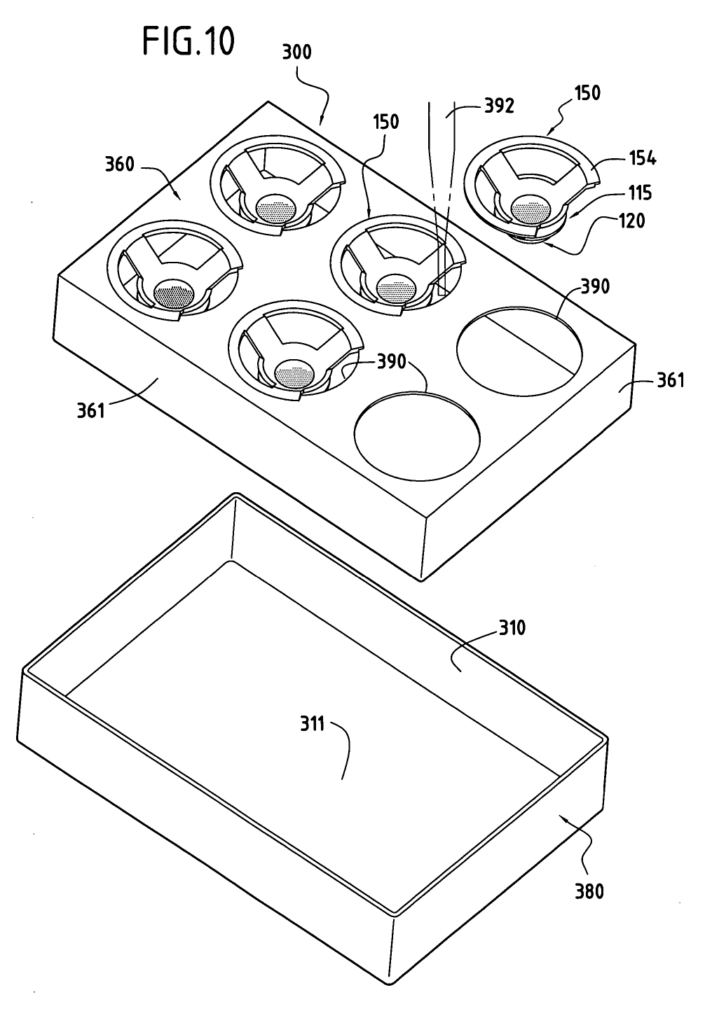

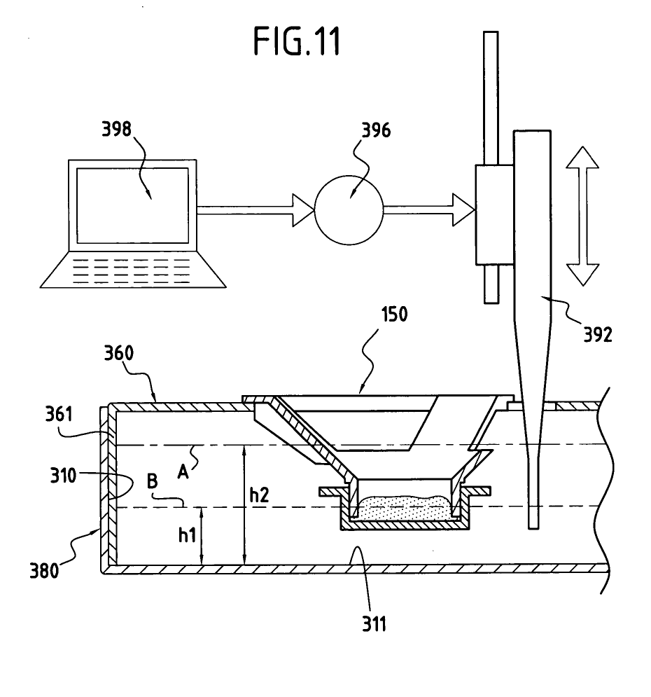

Furthermore, according to a second aspect, the invention relates to the use of the device as defined above on a roboticizable or automatable platform, as shown in FIGS. 10 and 11, preferably enabling the substrates to be positioned so that a device 392 for injecting or for aspirating the culture medium can be placed automatically, notably by being commanded by a robot or an automaton 396, e.g. itself commanded by a computer 398.

According to another advantageous feature of the invention, at least one part of the internal wall in contact with the culture medium, whether this be of the well and/or of the cradle and/or of the insert, is treated physically or chemically or biologically, or a combination of these, for promoting the cell culture, e.g. with a coating which promotes the adhesion and/or the proliferation of the cells.

In this context, it will be possible to use any physical or chemical method which modifies the overall ionic charge of the material constituting the internal wall in contact with the culture medium, advantageously a plastic material and/or a material which leads to the realisation of a coating of said internal wall with any biological molecule which promotes the adhesion of the culture medium and/or of the cells, such as collagen, fibronectin, laminin, etc.

In the introductory part of the present description, examples of material are given which can be used for producing this wall.

With reference to FIGS. 10 and 11, representation has been made of a fourth embodiment of a cell culture device according to the invention, which is represented by the general reference number 300, this comprising a single receptacle element 380 which constitutes a single common culture well 310 thus comprising a common culture bottom 311 intended to receive the culture medium, into which receptacle can be inserted a plurality of cradles or inserts, such as, for example, the cradles or inserts 150 of the embodiment of FIGS. 5 to 7, or 220-250 of the embodiment of FIG. 9.

Advantageously, with the view to maintaining the cradles or inserts 150 or 220-250 in a stable position, a lid 360 is provided according to the invention which is provided with as many orifices 390 as cradles or inserts provided. Thus, each orifice 390 can enable receiving and maintaining in position a cradle or insert 150 or 220-250.

It is understood that by virtue of this embodiment comprising a receptacle 380 forming a common culture well 310-311, by a single operation, it is possible to insert a tubular part of a pipette or point of a multi-channel pipette, such as that represented schematically in FIG. 10 bearing the reference number 392, and that is seen better in FIG. 11, or, similarly, of a cannula, for the automatic pouring in or removing, enabling the pouring in or removing of a solution of culture medium in the element forming a common well 310-311, this culture medium being represented schematically by the reference number 394 in FIG. 11.

The person skilled in the art understands easily that the pipette 392 can make up a part of a platform which is roboticizable or automatable and which is commanded by a robot or an automaton 396, e.g. itself commanded by a computer 398.

Within the context of the invention, it is possible to make successive cultures.

Thus, with a culture medium attaining level A represented in FIG. 11, it is for example possible to make a culture for a period of time of the duration of a cell culture.

Then, by pipetting, by pipette 392, it is possible to easily change, in one time, the culture medium and to introduce a volume of culture medium at a different level, e.g. level B, or conversely, here enabling putting the cells at the air/liquid interface.

With level A, a height h1 of liquid medium is obtained and with level B, a height h2 of liquid medium is obtained, this height being controlled by the volume of culture medium introduced into the common well 310, 311 formed by the receptacle 380 or bottom plate.

In the embodiment represented in FIG. 11, the lid 360 has its side edges 361 which come to penetrate into the inside of the receptacle 380, but provision can obviously be made that the side edge 361 of the lid 360 come to the outside of the receptacle 380.

According to an advantageous embodiment of the invention, the layer 36 of porous material is obtained in situ by pouring in a gel comprising collagen. It will be possible for this collagen to be of any origin and will advantageously be of bovine, porcine, equine or marine origin.

According to a particular embodiment, the gel comprises a mixture of collagen and at least one polysaccharide. It will be possible to use any type of polysaccharide and particularly a glycosaminoglycan, such as chondroitin 4-sulphate, chondroitin 6-sulphate, hyaluronic acid and their mixtures.

According to another particular embodiment, the gel comprises a mixture of collagen, at least one polysaccharide and chitosan, which is optionally modified. As modified chitosan, it will be possible to refer to the description of the closest document cited in the introductory part of the description, namely the patent EP 0 296 078, and which is incorporated herein in its entirety by reference.

The layer 36 of porous material can thus be made from the composition of a gel of a mixture of collagen, at least one polysaccharide and chitosan, as described in Example 1 of European Patent EP 0296 078.

However, in a non-obvious way according to the invention, the gel is poured:

a) either directly into the well 20, FIGS. 1 to 4,

b) or directly into the well 120 of the cradle 115, FIGS. 4 to 8,

c) or, even, directly into the cradle or insert 215, FIG. 9;

then, the dehydration of this gel thus poured in is carried out in situ in the well 20, or the bottom of the cradle or insert 120 or 215, preferably by freeze-drying or by heat dehydration, directly in the dehydration apparatus, preferably a freeze-dryer or an oven, optionally under vacuum.

Within the context of the invention, the well 20 or the bottom 120 of the cradle 115 or the insert 215 comprises as from the start, by this in situ preparation, the layer 36 of porous material serving as support of the culture medium which has no more need to be cut out and/or transferred from an intermediate heat dehydration or freeze-drying device to the culture well.

Further, by virtue of the means of maintaining in position, 50; 150; 250 respectively, of the peripheral edge 36a of the layer 36 of porous material, an anti-retraction effect is obtained of the peripheral edge 36a of the layer of porous material 36, as well as simultaneously, advantageously, a watertightness at the interface of the wall of the culture well, i.e. at the level of the peripheral edge 36a of the layer 36 of porous material, by obtaining the technical advantages set forth above.

The invention thus comprises all the technical means which are equivalent to the technical means described and represented in the annexed Figures which make up an integral part of the invention, as well as their various combinations.

What is claimed is:

1. A cell culture device, comprising at least one cell culture well intended for receiving cells with their culture medium, said device comprising at least one cell-supporting layer of porous material, this layer being obtained by dehydration of an aqueous gel which is poured directly either into said well bottom, or into the bottom of a cradle or insert of a size which is adapted for being insertable into the volume of the well.

2. The device of claim 1, wherein the dehydration is carried out by freeze-drying or by heat dehydration.

3. The device of claim 1, wherein at least the bottom of the well or of the cradle or insert containing the layer of porous material is sterilized after said dehydration, in a watertight packaging.

4. The device of claim 1, wherein the complete culture device is sterilized, in a watertight packaging.

5. The device of claim 3, wherein the sterilization is selected from the group consisting of a sterilization by irradiation with beta or gamma rays, and a sterilization by treatment with a sterilizing gas.

6. The device of claim 1, comprising an element maintaining in position at least the peripheral edge of the layer of porous material.

7. The device of claim 6, wherein said position maintaining element is ensuring watertightness at the peripheral interface of said layer of porous material and of the internal side wall facing it at the well bottom.

8. The device of claim 6, wherein the position-maintaining element is of a size which is sufficient to take support on said peripheral edge of the layer of porous material and to ensure the watertightness at said peripheral interface.

9. The device of claim 1, wherein said bottom of the well or of the cradle is detachable and joinable to the well.

10. The device of claim 1, wherein said bottom of the well or of the cradle is joined by fitting with slight forcing or snap-engagement to the well, ensuring the watertightness.

11. The device of claim 1, wherein said bottom of the well or of the cradle is joined to the outside of the well or of the cradle, the lower edge of the side wall of the well or of the cradle constituting an element maintaining in position the peripheral edge of said layer of porous material.

12. The device of claim 1, wherein at least one part of the internal wall of the bottom or of the cradle is treated by a treatment for promoting the cell culture selected from a physical treatment, a chemical treatment, a biological treatment, and any combination of same.

13. The device of claim 12, wherein the internal wall is treated with a coating selected from a coating which promotes the adhesion and a coating which promotes the proliferation of the cells.

14. The device of claim 1, comprising a plurality of cradles or inserts per culture well

15. The device of claim 14, comprising a lid which is provided with as many orifices as cradles or inserts, each orifice enabling receiving and maintaining in position a cradle or insert.

16. The device of claim 1, wherein the aqueous gel comprises collagen.

17. The device of claim 1, wherein the aqueous gel comprises a mixture of collagen and at least one polysaccharide.

18. The device of claim 1, wherein the aqueous gel comprises a mixture of collagen, at least one polysaccharide and/or chitosan.

19. A roboticizable or automatable platform which comprises a device as claimed in claim 1.

20. The platform of claim 19, wherein said platform is constructed to position the substrates to allow an automatic placing of a device for injecting, or for aspirating, of culture medium in, or from, said cell culture device,

21. The platform of claim 20, where in said platform is commanded by a robot or an automaton itself commanded by a computer.
